(19) United States
(12) Patent Application Publication
(10) Pub. No.: US 2009/0041741 A1
(43) Pub. Date: Feb. 12, 2009

(54) MODIFIED ENZYME AND TREATMENT METHOD

(75) Inventors: William S. Sly, St. Louis, MO (US); Jeffrey H. Grubb, Arnold, MO (US); Carole A. Vogler, St. Louis, MO (US)

Correspondence Address: HUSCH BLACKWELL SANDERS LLP, 720 OLIVE STREET, SUITE 2400, ST. LOUIS, MO, 63101, US

(73) Assignee: SAINT LOUIS UNIVERSITY, ST. LOUIS, MO (US)

(21) Appl. No.: 12/042,601

(22) Filed: Mar. 5, 2008

Related U.S. Application Data
(60) Provisional application No. 60/893,334, filed on Mar. 6, 2007.
(60) Provisional application No. 61/025,196, filed on Jan. 31, 2008.

(51) Int. Cl. A61K 38/45; C12N 9/24; A61K 38/47; C12N 9/00; A61P 43/00; C12N 9/16; C12N 9/10

(52) U.S. Cl. 424/94.5; 435/200; 424/94.61; 435/183; 435/196; 435/193; 435/201; 424/94.6

(57) ABSTRACT

There is disclosed an isolated, modified recombinant β-glucuronidase wherein the modification is having its carbohydrate moeties chemically modified so as to reduce its activity with respect to mannose and mannose 6-phosphate cellular delivery system while retaining enzymatic activity Also disclosed are methods for the treatment of lysosomal storage disease in mammals wherein the mammal is administered a therapeutically effective amount of isolated, modified recombinant β-glucuronidase whereby said storage diseased is relieved in the brain and visceral organs of the mammal. Also disclosed are other lysosomal enzymes within the scope of the invention.

### CROSS-REFERENCE TO RELATED APPLICATIONS

This application claims the benefit of the priorities of U.S. Provisional Patent Application No. 60/893,334 filed Mar. 6, 2007, and U.S. Provisional Patent Application No. 61/025,196, filed Jan. 31, 2008. The disclosures of each of the foregoing applications are hereby incorporated by reference in their entirety.

### FIELD OF THE INVENTION

This invention relates to an improved enzyme, β-glucuronidase, having an improved half-life in the circulation of a mammal such that the treatment of mucopolysacharridosis is improved by intravenous infusion of the mammal with said enzyme.

### BACKGROUND OF THE INVENTION

Many mucopolysacharridosis (MPSw) disorders, including MPS VII, show evidence of significant storage of glycosaminoglycans in the lysosomes of different cell types in the brain as well as in the visceral organs (1). The currently accepted treatment for some of these diseases, referred to as enzyme replacement therapy (ERT) relies on intravenous infusion of recombinant enzyme into the patient. This method of treatment has successfully cleared storage material from visceral organs and resulted in clinical improvement in these lysomal storage diseases (LSDs)(2-5). Unfortunately in these cases little to no infused enzyme has been able to cross the blood brain barrier (BBB) so limited or little improvement has been achieved in the central nervous system (CNS) (6).

When enzyme was infused into newborn mice, considerable enzyme was delivered to brain, and CNS storage was reduced (7-9). However, brain storage was resistant to clearance if ERT was begun after 2 weeks of age. Recent studies indicated that this enzyme delivery to the CNS in the newborn period was caused by mannose 6-phosphate receptor (M6PR)-mediated transcytosis (10). Down-regulation of this receptor by age 2 weeks appeared to explain the resistance of brain to ERT in the adult. Recently, efforts were made to improve the delivery of β-glucuronidase to the brain in the MPS VII mouse model (11). These studies have shown that increasing the dose of enzyme, which results in slower clearance from the circulation, slightly enhanced the delivery to the brain (12-14). Also infusing mice deficient in the mannose receptor increased the amount of time the enzyme stayed in the circulatory system (15). To account for enzyme delivery to adult brain, it was speculated that increasing the enzyme dose saturated the clearance receptors and slowed clearance of the enzyme from the circulation, resulting in more delivery to the brain (11, 15), or clearing CNS storage after multiple infusions of large doses of corrective enzyme (12-14).

Whether the high circulating levels of enzyme were required for delivery by receptors that were less abundant in adults than neonates or exposure to high circulating levels of enzyme led to delivery by another route is an important question. To address this question, we analyzed ERT in MPS VII mice that were mannose receptor (MR)-deficient (15). When GUS was infused into MR-deficient MPS VII mice, the enzyme clearance was indeed prolonged, although considerably less than expected, because of efficient clearance by hepatic M6PR (11, 15).

### BRIEF DESCRIPTION OF THE DRAWINGS

FIG. 1, A and B, is the Gus insert (A) and the mammalian expression vector pCXN (B) into which it was cloned (29).

FIG. 2 is a graphical representation of the data obtained in Example 2 showing stability data of GUS and PB-GUS at 65° C.

FIG. 3 is a graphical representation of the data obtained in Example 2 showing stability data of GUS and PB-GUS at 37° C. in the lysosomes of human fibroblasts.

FIG. 4 is a graphical representation of data obtained in Example 3 showing the clearance of GUS and PB-GUS from plasma of ERT treated mice as a function of time.

FIG. 5 is a collection of photomicrographs of brain tissue of GUS- and PB-GUS-treated mice showing neuronal and meningeal storage of lysomal tissue after treatment in accordance with the procedure of Example 5.

FIG. 6 is a graphical representation of data obtained in Example 5 showing the number of vacuoles of lysosomal storage per 500 cortical neurons in brains of mice treated with GUS and PB-GUS.

### SUMMARY OF THE INVENTION

Novel modified lysosomal enzymes and methods of their use in the treatment of mammals afflicted with LSDs have now been discovered. Such modified enzymes have increased half-life in the circulatory system resulting in improved treatment of LSDs. Such modification chemically inactivates the oligosaccharides on the lysosomal enzymes thereby inactivating traditional recognition markers on the enzyme that mediates their rapid clearance from the circulation system as will be further described below.

In order to slow down the clearance of β-glucuronidase after infusion into the circulatory system of a mammal, the oligosaccharides on the glycoprotein are chemically inactivated by treating the β-glucuronidase sequentially with sodium-meta-periodate and sodium borohydride. This treatment inactivates the two traditional recognition markers on the enzyme that mediate its rapid clearance from the circulation by means of the mannose and mannose 6-phosphate receptors. This in effect increases the half-life in the circulation from 11 minutes for the untreated enzyme (GUS) to 18.5 h for the periodate/borohydride treated enzyme (PB-GUS, also known in the art as PerT-GUS). The efficacy of these enzymes was determined in a 12-week ERT experiment in which MPS VII mice were treated with weekly infusions of GUS vs. PB-GUS at doses of 0, 2 mg/kg and 4 mg/kg body weight. A slight improvement was observed in the amount of storage material in the cortical neurons in the brains of mice treated with 4 mg/Kg. There was a remarkable clearance of 95% of storage from the cortical neurons in the brains of mice treated with both 2 mg/kg and 4 mg/kg of PB-GUS. Also, there was observed significant continued clearance of storage material from the visceral organs from mice treated with both types of enzyme at both doses of 2 and 4 mg/kg body weight.

These results seem to indicate that slowing the clearance and maintaining high concentrations of β-glucuronidase in the circulation after infusion facilitates delivery of the enzyme across the BBB by some mechanism. Since the mannose and mannose 6-phosphate delivery systems have been inactivated as a result of the periodate treatment, this delivery must be mediated by some other method. One possible method would be by increased fluid-phase pinocytosis, a mechanism that would be greatly enhanced by maintaining high levels of enzyme present for long periods of time in the circulation. Whatever the mechanism is, use of the periodate-treated enzyme shows great promise for treating the brain in MPS VII and any of the other lysosomal storage diseases where there is brain pathology. This method may also be extended for use for other glycoproteins where rapid clearance from the circulation by the mannose or mannose 6-phosphate delivery systems hinders their therapeutic effect.

Accordingly, in one aspect the invention is directed to a composition useful in enzyme replacement therapy, the composition comprising a lysosomal storage enzyme treated with a chemical to inactivate carbohydrate moieties on the enzyme, such that the lysosomal enzyme is not readily taken up by a target cell by the mannose and mannose 6-phosphate delivery systems. A preferred chemical-to-inactivate is a periodate followed by treatment with a borohydride. A preferred NPS enzyme is β-glucuronidase. It is preferred to employ any suitable alkali metal periodate and alkali metal borohydride. The preferred alkali metal is sodium.

In another embodiment, the invention is directed to a method of treating a patient having a lysosomal storage disease comprising administering to the patient a therapeutically effective amount of a composition comprising a medically suitable excipient and a lysosomal enzyme treated with a chemical to inactivate carbohydrate moieties on the enzyme, such that the enzyme is not readily taken-up by a target cell by the mannose and mannose 6-phosphate delivery systems. A preferred treatment is with a periodate followed by treatment with sodium borohydride. A preferred MPS enzyme is β-glucuronidase which is effective to treat lysosomal storage disease preferably MPS VII (Sly syndrome).

### DETAILED DESCRIPTION OF THE INVENTION

In summary, there has been discovered a means to successfully treat GUS with periodate and borohydride without significantly reducing the enzymatic activity or stability. The treated protein has been shown to have modified carbohydrate that no longer has functional recognition signals for mannose and mannose 6-phosphate receptors. Because of this, the enzyme exhibits a vastly increased half-life in the circulation after intravenous infusion. This increased availability results in the improved delivery of the enzyme across the BBB by some unknown mechanism. Whether it is increased opportunity for fluid phase pinocytosis or some other “leakiness”, the enzyme, once it has crossed the BBB, has increased access to cells in the brain. It is then able to use its enzymatic activity to clear accumulated storage material in the cells and hopefully reverse the progression of the disease MPS VII.

While not wishing to be bound by any particular theory, the use of periodate treated enzyme shows great promise for treating the brain in MPS VII and any of the other lysosomal diseases where there is brain pathology. This method can reasonably be extended for use with other glycoproteins where rapid clearance from the circulation hinders their therapeutic effect. Any number of lysosomal enzymes are included within the scope of this invention. Examples of such enzymes are heparin N-sulfatase for treatment of MPS III (Sanfillipo A), hexosaminidase A for treatment of Tay-Sachs disease, α-L-iduronidase for treatment of MPS I Hurler Syndrome), palmitoyl thiotransferase (PPT1) for Batten's disease (CLN1), α-glucosidase for Pompe disease, N-acetyl-galactosamine-6-sulfatase for MPS IVA and β-galactosidase for MPS IVB (Morquio disease A and B), and N-acetylgalactosamine 4-sulfatase for MPS VI (Maroteaux-Lamy syndrome). Other enzymes can be easily envisioned by those of ordinary skill in view of this disclosure and are included within the scope of this invention. The enzymes disclosed herein when modified in accordance with this invention are therapeutically effective to treat various diseases. The effective amount of such modified enzymes can be easily determined by simple testing. However the term “effective amount” as used herein is intended to mean that amount which will be therapeutically effective to treat the disease. Such amount is generally that which is known in the art for the use of such enzymes to therapeutically treat known diseases.

### Generation of Stable Cell Lines Secreting GUS

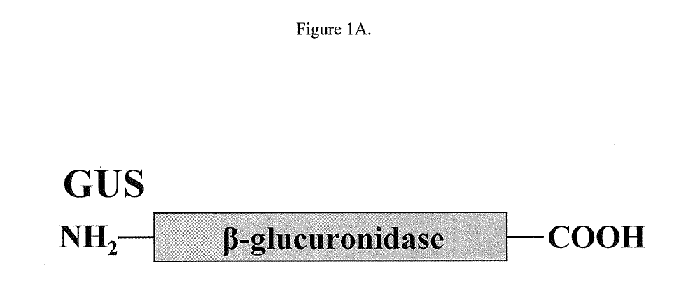

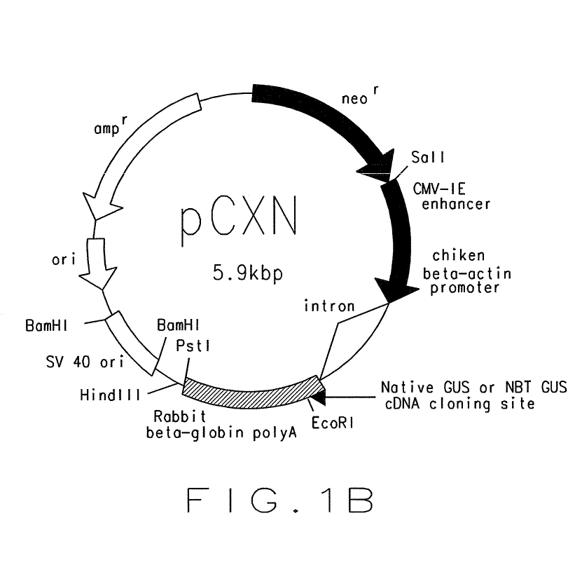

Using DNA cloning techniques, the cDNA sequence encoding the full length cDNA for human β-glucuronidase was subcloned (Genbank Accession # NM_{—}000181) (FIG. 1) into the mammalian expression vector pCXN (29). This expression vector contains an expression cassette consisting of the chicken beta-actin promoter coupled to the CMV Intermediate-early (CMV-IE) enhancer. pCXN also contains a selectable marker for G418 allowing selection of stably expressing mammalian cells SEQ ID NO. 1.

This plasmid was introduced into the Chinese hamster ovary cell line, CHO-K1(34) by electroporation (30). After selection in growth medium consisting of Minimal Essential Medium+35 μg/ml proline+15% fetal bovine serum (FBS)+400 μg/ml G418, colonies were picked and grown to confluency in 48-well plates. High level expressing clones were identified by measuring GUS activity secreted into the conditioned medium from these clones. The highest-producing clone was scaled up and secreted enzyme was collected in protein-free collection medium PF-CHO. Conditioned medium collected in this way was pooled, centrifuged at 5000×g for 20 min and the supernatant was collected and frozen at 20° F. until sufficient quantities were accumulated for purification.

### Measurement of GUS Activity

GUS activity was measured using the 10 mM 4-methyl-umbelliferyl β-D-glucuronide as substrate in 0.1M sodium acetate buffer pH 4.8, 1 mg/ml crystalline BSA as previously described (31).

### Purification of GUS

β-glucuronidase was purified by two different methods. The first method was by a multi-step procedure using conventional column chromatography. The second method utilized an anti-human β-glucuronidase monoclonal antibody affinity resin followed by a desalting step. The complete procedures for both methods are outlined below.

### Conventional Purification

A: Ultrafiltration: YM-100 membrane; Diafiltrate with 20 mM NaPO₄+150 mM NaCl+0.025% NaN₃ @ pH 5.5; (2×2.25 L).

B: Blue Sepharose FF(GE Healthcare): Equilibrate 10× column volume column with 20 mM NaPO₄ @ pH 5.5; Load concentrate from ultrafiltration (don't adjust pH, range: 5.5-5.7); Wash 10× column volume with 20 mM NaPO₄+150 mM NaCl @ pH 5.5; Elute column with 10 mM NaPO₄+800 mM NaCl @ pH 7.5; Regeneration: Wash with 10× column 20 mM NaPO₄ @ pH 5.5+2M NaCl.

C: Phenyl Sepharose (High Sub FF): Equilibrate 30× column volume with 10 mM NaPO₄+1000 mM NaCl @ pH 8.0; Load pooled blue elute as is (don't adjust pH, range: 7.2-7.4); Wash 10× column volume with 10 mM NaPO₄+1000 mM NaCl @ pH 8.0; Elute column with 10 mM Tris+1 mM Na-β-Glycerophosphate @ pH 8.0; Dialyze elution with 3 changes of 10 mM Tris+1 mM Na-β-glycerophosphate @ pH 8.0; Regeneration: Wash with 0.5 M NaOH, 30 min contact time; Wash with 30 column volumes of ddH₂O.

D: DEAE Sephacel: Equilibrate 10× column volume with 10 mM Tris+1 mM Na-β-glycerophosphate @ pH 8.0; Load pooled dialyzed Phenyl elute. Wash 10× column volume with 10 mM Tris+1 mM Na-β-glycerophosphate @ pH 8.0; Elute with 0-0.4M NaCl gradient; Dialyze DEAE pooled eluate in 25 mM Na Acetate+1 mM Na-β-glycerophosphate; +0.025% NaN₃ @ pH 5.5; Regeneration: Wash with 20× column volume 10 mM Tris+1 mM Na-β-glycerophosphate @ pH 8.0+2 M NaCl.

E: CM Sepharose: Equilibrate lOx column volume with 25 mM Na Acetate+1 mM Na-β-Glycerophosphate+0.025% NaN₃ @ pH 5.5; Load dialyzed DEAE pooled eluate; Elute with 0-0.3M NaCl gradient. Regeneration: Wash with 20× column volume 25 mM Na Acetate+1 mM Na-β-Glycerophosphate+0.025% NaN₃ @ pH 5.5+2M NaCl.

### Monoclonal Purification

Affinity chromatography procedure was performed essentially as follows: Conditioned medium from CHO cells overexpressing the GUS protein was filtered through a 0.22μ filter. Sodium chloride (crystalline) was added to a final concentration of 0.5M, and sodium azide was added to a final concentration of 0.025% by adding 1/400 volume of a 10% stock solution. The medium was applied to a 5 ml column of anti-human β-glucuronidase-Affigel 10 (pre-equilibrated with Antibody Sepharose Wash Buffer: 10 mM Tris pH 7.5, 10 mM potassium phosphate, 0.5 M NaCl, 0.025% sodium azide) at a rate of 25 ml/h at 4° C. The column was washed at 36 ml/h with 10-20 column volumes of Antibody Sepharose Wash Buffer. The column was eluted at 36 ml/hour with 50 ml of 10 mM sodium phosphate pH 5.0+3.5 M MgCl₂. Fractions of 4 ml each were collected and assayed for GUS activity. Fractions containing the purifed protein were pooled, diluted with an equal volume of P6 buffer (25 mM Tris pH 7.5, 1 mM β-glycerophosphate, 0.15 mM NaCl, 0.025% sodium azide) and desalted over a BioGel P-6 column (pre-equilibrated with P6 buffer) to remove the MgCl₂ and to change the buffer to P6 buffer for storage. GUS protein was eluted with P6 buffer, fractions containing GUS activity were pooled and the final pool assayed for GUS activity and protein. Purified GUS was stored frozen at −80° C. in P6 buffer for long-term stability. For mouse infusions, the enzymes were highly concentrated in Centricon YM-30 concentrators and the buffer was changed to P6 Buffer without azide. These concentrates were frozen in small aliquots at −80° C. until use.

### Characterization of Purified GUS

GUS is a 300 kDa protein that exists as a homotetramer consisting of four identical monomers of apparent molecular weight of 75 kDa. The purified recombinant GUS used in these experiments was similar to that described (11, 19). The apparent molecular mass of the enzyme monomer was 75 kDa on reducing SDS-PAGE. The tetrameric enzyme had a molecular mass of ≈300 kDa when analyzed by sizing gel filtration chromatography (data not shown). The specific activity of the purified enzyme was 5.0×10⁶ units/mg. The Kᵤₚₜₐₖₑ was 1.25-2.50 nM, calculated from uptake saturation curves by using human MPS VII fibroblasts in which the uptake is almost entirely M6PR-dependent. To confirm molecular weight, 2 and 4 μg of purified GUS were analyzed by SDS-PAGE under reducing conditions (35). The apparent molecular weight was 75 kDa as expected.

The following examples are presented to illustrate the instant invention and are not meant to limit the scope of the invention to these particular examples. The skilled artisan, in the practice of this invention, will readily and reasonably understand that the methods and compositions are applicable to any and all enzymes and proteins that gain entry into a cell via the mannose and mannose 6-phosphate pathways.

### EXAMPLE 1

### Treatment of Purified GUS with Periodate and Borohydride

The mannose and manose 6-phosphate recognition sites on GUS are both located in the carbohydrate portion of GUS enzyme. In order to inactivate this carbohydrate moiety, the enzyme was treated by a well established procedure utilizing reaction with sodium meta-periodate followed by sodium borohydride (17, 18). Approximately 10 mg of purified GUS was treated with a final concentration of 20 mM sodium meta-periodate in 20 mM sodium phosphate, 100 mM NaCl pH 6.0 for 6.5 h on ice in the dark. The reaction was quenched by the addition of 200 mM final concentration ethylene glycol and incubated for an additional 15 min on ice in the dark. Afterwards, this mixture was dialyzed against 2 changes of 20 mM sodium phosphate, 100 mM NaCl pH 6.0 at 4° C. The periodate treated, dialyzed enzyme was then treated with the addition of 100 mM final concentration sodium borohydride overnight on ice in the dark to reduce reactive aldehyde groups. After this treatment, the enzyme was dialyzed against two changes of 20 mM sodium phosphate, 100 mM NaCl, pH 7.5 at 4° C. The final dialyzed enzyme was stored in this buffer at 4° C. where it was stable indefinitely.

### Characterization of the Periodate and Borohydride Treated GUS

Treatment of GUS with periodate and borohydride resulted in only a slight inactivation of the enzymatic activity. The specific activity prior to treatment was 5.0×10⁶ units/mg and following treatment was 4.5×10⁶ units/mg.

To assess the effectiveness of the periodate and borohydride treatment in inactivating the carbohydrate on the enzyme, the ability of the enzyme to be taken up by human β-glucuronidase deficient fibroblasts or by the permanent J774E mouse macrophage line was analyzed. M6PR-mediated uptake was determined by adding 4,000 units of GUS or PB-GUS±2 mM M6P in 1 ml of growth medium to 35-mm dishes of confluent GM-2784 GUS-deficient fibroblasts. After incubation at 37° C. and 5% CO₂ for 2 h, the cells were cooled on ice, washed five times with cold PBS, then solubilized in 0.5 ml of 1% sodium deoxycholate. Extracts were assayed for GUS activity and protein. Values were expressed as units of enzyme taken up per mg of cell protein per hour of uptake.

MR-mediated uptake was measured by adding 10,000 units of GUS or PB-GUS±1.7 mg/ml yeast mannan (Sigma-Aldrich) in 1 ml of growth medium to 35-mm dishes of confluent J774E mouse macrophages (33). After incubation at 37° C. and 5% CO₂ for 4 h, the cells were washed as above and then solubilized in 1 ml of 1% sodium desoxycholate and assayed for GUS activity.

Table 1 below shows the M6P-receptor mediated uptake of untreated or mock-treated GUS by the human fibroblast cell line. GUS is taken up by this line at the rate of 377 units/mg cell protein/1 h of uptake. Two mM M6P completely inhibits this uptake. In contrast, the uptake of the periodate and borohydride treated GUS(PBGUS) has been completely destroyed. Table 2 below shows that untreated GUS is taken up by the mouse macrophage line at a rate of 316 u/mg cell protein/1 h of uptake and the uptake is inhibited by the presence of 1.69 mg/ml yeast mannan. In contrast, three separate batches of periodate and borohydride treated GUS(PBGUS) have essentially no uptake by this cell line.

TABLE 1
FIBROBLAST UPTAKE ON HBG 5-6 +/− PERIODATE
AND BOROHYDRIDE TREATMENT

| | Uptake | M6P-Specific Uptake |
|---|---|---|
| Condition | u/mg/1 h | u/mg/1 h |
| GUS | 380 | 377 |
| GUS + 2 mM M6P | 3 | — |
| GUS Mock Treated | 363 | 359 |
| GUS Mock Treated + 2 mM M6P | 3.5 | — |
| PB-GUS Periodate&Borohydride | 1 | 0 |
| Treated | | |
| PB-GUS Periodate&Borohydride | 1 | — |
| Treated + 2 mM M6P | | |

TABLE 2
J774E MACROPHAGE UPTAKE ON HBG 5-6 +/− PERIODATE
AND BOROHYDRIDE TREATMENT

| | Uptake | Man-Specific Uptake |
|---|---|---|
| Condition | u/mg/1 h | u/mg/1 h |
| GUS | 366 | 316 |
| GUS + 1.69 mg/ml Yeast Mannan | 50 | — |
| PB-GUS | 8 | 3 |
| PB-GUS + Yeast Mannan | 5 | — |
| PB-GUS | 11 | 2 |
| PB-GUS B34E + Yeast Mannan | 9 | — |
| PB-GUS | 12 | 0 |
| PB-GUS + Yeast Mannan | 21 | — |

Since both mannose 6-phosphate and mannose receptor mediated uptake are dependent on functional mannose 6-phosphate or mannose residues, respectively, these results indicate that the periodate and borohydride treatment of GUS (PB-GUS) has inactivated the carbohydrate structures on the enzyme.

### EXAMPLE 2

### Stability of Native GUS or PB-GUS

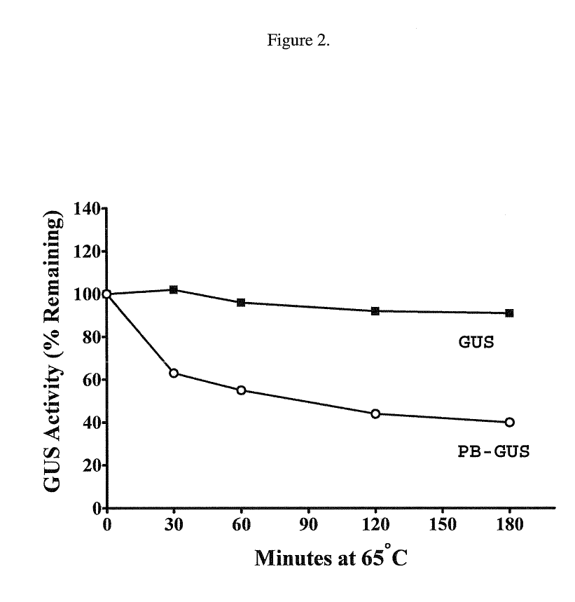

The carbohydrates on glycoproteins often confer enhanced thermal stability, and removal of oligosaccharide chains often destabilizes glycoproteins (21). Human GUS has been shown to be relatively stable to thermal inactivation at 65° C. (22-26). Purified GUS or PB-GUS was diluted in equal volumes of heat inactivation buffer [40 mM Tris-HCl (pH 7.5), 150 mM NaCl, 10 mg/ml BSA], and aliquots were incubated for 0, 0.5, 1, 2, or 3 h at 65° C. After treatment, aliquots were cooled on ice and then assayed for GUS activity. Results were expressed as the percentage of original units of GUS activity remaining at the indicated times. As shown in FIG. 2, recombinant GUS retained 90% of initial activity after 3 h at 65° C., whereas PB-GUS retained 40% of its activity under these conditions (FIG. 2).

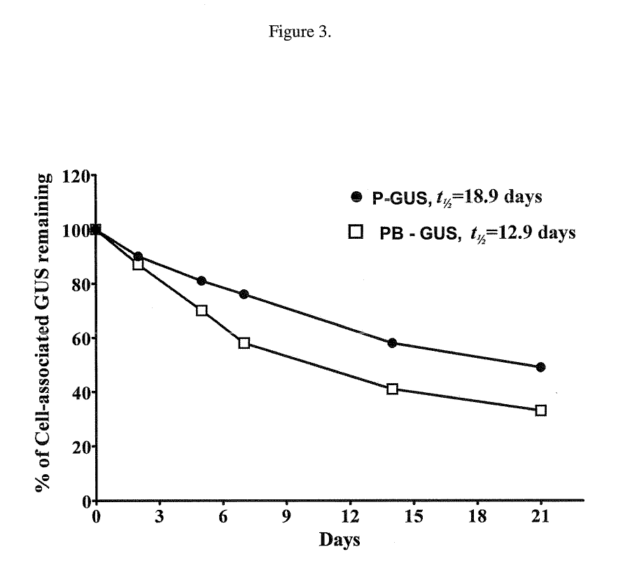

To compare the stability of GUS and PB-GUS in lysosomes of living cells at 37° C., a study was conducted to determine their half-life after uptake by MPS VII fibroblasts. The low rate of endocytosis of PB-GUS by fibroblasts required exposure to 100,000 units/ml PB-GUS per plate for 48 h to accumulate sufficient enzyme by fluid phase pinocytosis (28 units per plate) to allow measurement of its half-life. By contrast, fibroblasts exposed to 500 units/ml M6P containing native GUS for 48 h contained 228 units per plate. Tissue culture dishes (35 mm) of confluent GM-2784 GUS-deficient fibroblasts were incubated with 500 units of GUS or 100,000 units of PB-GUS in 1 ml of growth medium at 37° C. and 5% CO₂ for 48 h under sterile conditions. The plates were washed twice with sterile growth medium and then fed with 2 ml of the same. Duplicate plates were taken off at 0, 2, 5, 7, 14, and 21 days, washed five times with PBS and frozen at −20° C. Remaining plates were fed twice weekly with 2 ml of growth medium. After all plates had been collected, the cells were solubilized in 0.5 ml of 1% desoxycholate and assayed for GUS activity. Values were expressed as percentage of zero time cell-associated GUS activity remaining at the indicated time points. FIG. 3 shows the half-life for the two enzymes in fibroblasts upon subsequent incubation at 37° C. The t_{1/2} of GUS was 18.9 days. The t_{1/2} of PB-GUS was shorter (12.9 days), but nearly one-third of the initial activity was still present at 21 days.

### EXAMPLE 3

### Clearance of the Periodate and Borohydride Treated GUS from the Circulation After IV Infusion

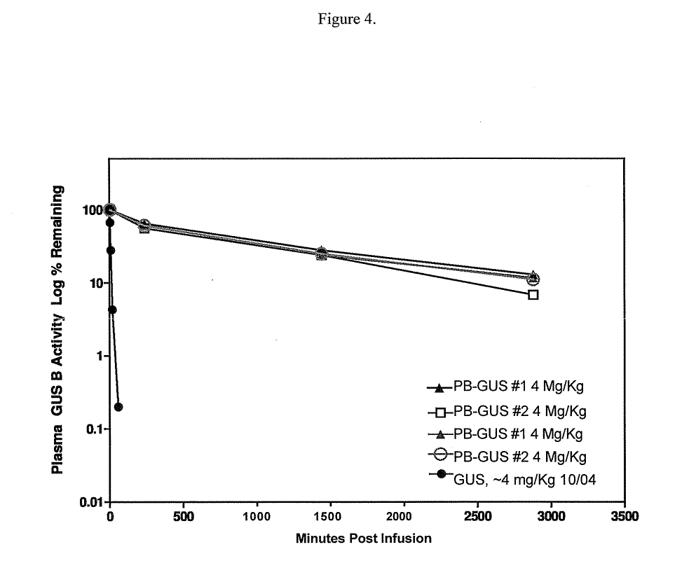

As stated previously, the purpose of treating GUS with periodate and borohydride, was to drastically slow its clearance time from the circulation after infusion. To test this, the tail veins of MPS VII mice were infused with GUS or PB-GUS at a dose of 4 mg/kg body weight in a total volume of 125 μl of PBS. After infusion, blood samples were taken by supraorbital puncture at 2, 5, 10, 20, 60, 90, and 120 min for GUS and 4, 240, 1,440, and 2,880 min for PB-GUS into heparinized capillary tubes. Plasma was collected after centrifugation and assayed for GUS activity. Values were expressed as a percentage of GUS activity remaining compared with the first time point. FIG. 4 and Table 3 below show the results of that clearance study. As can be seen, the clearance of untreated GUS is very rapid with a t_{1/2} of 11.7 min. In contrast, the clearance of PB-GUS in four separate mice was drastically slower with a t_{1/2} of 18.5±1.0 h. This would indicate that the rapid clearance of this enzyme due to the mannose and mannose 6-phosphate receptor (15) has been abrogated.

TABLE 3
CLEARANCE OF GUS AND PB-GUS FROM THE CIRCULATION
OF EAM MICE AFTER INFUSION WITH 4 MG/KG ENZYME

| | GUS | | PB-GUS #1 | | PB-GUS #2 | | PB-GUS #3 | | PB-GUS #4 | |
|---|---|---|---|---|---|---|---|---|---|---|
| Min. | u/ml | % | u/ml | % | u/ml | % | u/ml | % | u/ml | % |
| 2 | 261,440 | 100 | | | | | | | | |
| 4 | — | — | 318,960 | 100 | 228,240 | 100 | 285,120 | 100 | 369,120 | 100 |
| 5 | 174,720 | 67 | | | | | | | | |
| 10 | 73,920 | 28 | | | | | | | | |
| 20 | 11,200 | 4.3 | | | | | | | | |
| 60 | 640 | 0.2 | | | | | | | | |
| 90 | 0 | 0 | | | | | | | | |
| 120 | 0 | 0 | | | | | | | | |
| 240 | | | 177,840 | 56 | 147,960 | 65 | 176,640 | 62 | 225,120 | 61 |
| 1440 | | | 75,240 | 24 | 64,440 | 28 | 68,640 | 24 | 94,080 | 25 |
| 2880 | | | 21,660 | 6.8 | 29,520 | 12.9 | 33,120 | 11.6 | 41,280 | 11.1 |

| | | | | | |
|---|---|---|---|---|---|
| t_{1/2} | 11.7 min | 1022 min | 1195 min | 1119 min | 1114 min |
| | 0.2 h | 17.0 h | 19.9 h | 18.6 h | 18.6 h |

Mean = 1113 ± 61 min
18.5 ± 1.0 h

### EXAMPLE 4

### Tissue Distribution of GUS vs. PB-GUS

Previously, the plasma clearance of the enzyme was observed to be slowed when treating MPS VII mice with high-dose GUS and facilitated enzyme delivery to the brain (11). In these experiments, it was not clear whether it was the higher dose of enzyme itself or the delayed plasma clearance of the enzyme that accounted for improved delivery to brain. To address this question, comparative measurements were made of the distribution of GUS and PB-GUS in brain and other tissues 48 h after infusion into MPS VII mice. Mice were perfused with Tris-buffered saline before collection of tissues to ensure that tissue was not contaminated with residual plasma enzyme. MPS VII mice were infused via tail vein with GUS or PB-GUS at a dose of 4 mg/kg in a total volume of 125 μl of PBS. At 48 h after infusion, the mice were perfused with 30 ml of 25 mM Tris (pH 7.2), 140 mM NaCl. Perfused tissues were collected and flash frozen in liquid nitrogen until further processing. Tissues were thawed, weighed, and homogenized for 30 s with a Polytron homogenizer in 10-20 volumes of 25 mM Tris (pH 7.2), 140 mM NaCl, 1 mM phenylmethylsulfonyl fluoride. Total homogenates were frozen at −80° C., thawed, and then sonicated for 20 s to produce a homogeneous extract. Extracts were assayed for GUS activity and protein, and the results were expressed as units/milligrams of tissue protein. The results of these measurements appear in Table 4 below.

TABLE 4
DISTRIBUTION IN BRAIN AND
TISSUE OF GUS AND PB-GUS

| | Wild-type | GUS | PB-GUS |
|---|---|---|---|
| | levels* | (4 mg/kg)^{†} | (4 mg/kg) |
| Tissue | (n = 4) | (n = 2) | (n = 3) |
| Brain | 16.7 ± 2 | 0.23 ± 0.005 | 1.30 ± 0.28 |
| Liver | 185 ± 11.9 | 892 ± 45.5 | 230 ± 63 |
| Spleen | 301 ± 26.6 | 558 ± 54 | 122 ± 51 |
| Heart | 20.8 ± 12.5 | 13.0 ± 1.8 | 44.1 ± 16.3 |
| Kidney | 108 ± 7.5 | 11.9 ± 0.19 | 21.7 ± 3.6 |
| Lung | ND^{‡} | 5.1 ± 0.4 | 19.9 ± 6.1 |
| Muscle | 4.95 ± 1.80 | 1.2 ± 0.07 | 6.3 ± 3.5 |
| Bone + marrow | 161 ± 35 | 75.6 ± 17 | 59.5 ± 24.8 |
| Eye | 4.88 ± 0.68 | 0.90 ± 0.52 | 4.9 ± 1.5 |

As is evident from the data in Table 4, delivery of native GUS to brain at 48 h was minimal. However, native GUS was delivered to other tissues at levels similar to those previously reported. PB-GUS was delivered to heart, kidney, muscle, lung, and eye at levels higher than those seen with native GUS. The levels in liver and spleen were nearly 4-fold lower after PB-GUS infusion than after GUS infusion. This result undoubtedly reflects the curtailment of receptor-mediated uptake by the MPR and M6PR that are highly expressed in these two tissues. By contrast, brain levels were greatly increased (7.8% of wild-type) in PB-GUS-infused animals. This result suggests that the long circulating PB-GUS has an advantage in crossing the BBB. Thus, it was of great interest to study its effectiveness in clearing storage from cells in the CNS.

### EXAMPLE 5

### Comparison of the Efficacy of Periodate/Borohydride Treated GUS for ERT in Clearing Neuronal Storage

As stated previously, it was believed that slowing the clearance of GUS from the circulation might facilitate the delivery to the brain. It has been shown above that the periodate and borohydride treatment accomplished this producing an enzyme with a much reduced rate of clearance from the circulation after IV infusion. The effectiveness of the treated enzyme in clearing the storage material from the lysosomes of the MPS VII mouse after a typical ERT regimen was tested. MPS VII mice were treated with 12 weekly infusions, one group with untreated GUS at doses of 2 or 4 mg/kg body weight and a second group with PB-GUS at doses of 2 or 4 mg/kg body weight. Two other groups of MPS VII mice were infused two times daily for 1 week with a total of 48 mg/kg, one group with GUS and one group with PB-GUS. One week after the last infusion, tissues from the group receiving untreated GUS (n=3), 2 mg/kg (n=3) or 4 mg/kg GUS (n=2), and PB-GUS, 2 mg/kg (n=2) or 4 mg/kg (n=3) were obtained at necropsy after Tris-buffered saline perfusion, fixed in 2% paraformaldehyde and 4% glutaraldehyde, post fixed in osmium tetroxide, and embedded in Spurr's resin. For evaluation of lysosomal storage by light microscopy, toluidine blue-stained 0.5-μm-thick sections of liver, spleen, kidney, brain, heart, rib, and bone marrow were assessed blind. To evaluate storage in cortical neurons, 500 contiguous parietal neocortical neurons were scored for the number of lucent cytoplasmic vacuoles, indicating lysosomal storage. A maximum of seven vacuoles were counted per cell, and results were evaluated by ANOVA or Student's t test. Also evaluated were the hippocampal neurons by counting the number of vacuoles in 100 neurons in CA2 sector. Other tissues were examined by using a semiquantitative scale, as described in ref. 11.

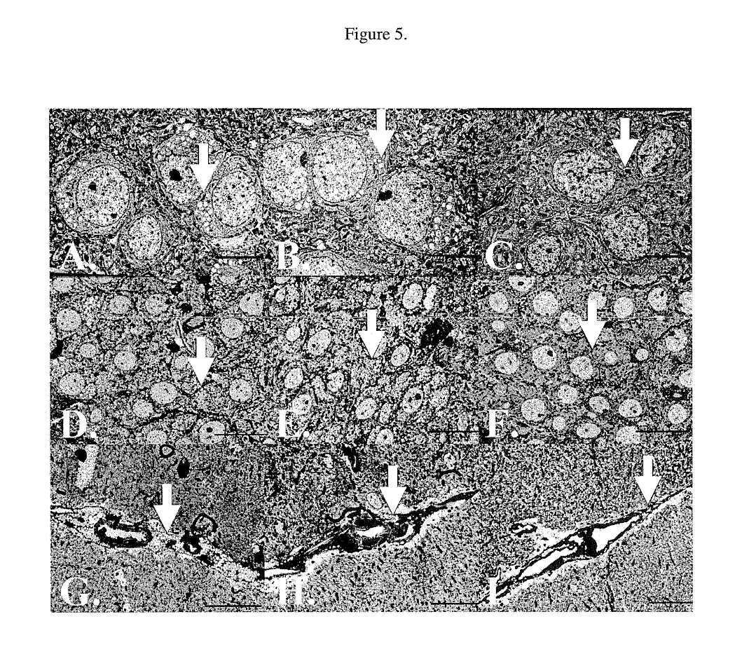

As can be seen in FIG. 5, GUS results in a slight reduction of the storage material in the brain whereas PB-GUS results in almost complete reversal of the storage. This would indicate that the periodate and borohydride treated GUS was vastly more effective in treating the brain storage in this disease.

In FIG. 5, reduction in neuronal and meningeal storage with ERT with GUS and PB-GUS is shown as follows: (A) Neocortical neurons from an untreated MPS VII mouse have abundant lysosomal storage in the cytoplasm (arrow). (B) After treatment with 4 mg/kg GUS, there is still a moderate amount of cytoplasmic storage (arrow) despite the therapy. (C) After 4 mg/kg PB-GUS, there is a marked reduction in the amount of storage in the neocortical neurons (arrow). (D) The CA2 sector hippocampal neurons have abundant storage (arrow) in untreated MPS VII mice. (E) After treatment with GUS, the amount of storage in neurons (arrow) the same area of the hippocampus is similar to that of the untreated mouse. (F) After treatment with PB-GUS, there is a remarkable reduction in the amount of storage in neurons (arrow) in the CA2 sector of the hippocampus. (G) The meninges of an untreated MPS VII mouse has abundant storage in fibroblasts around vessels (arrow). (H) Storage (arrow) is moderately decreased after treatment with GUS. (1) Treatment with PB-GUS also produces moderate reduction in storage (arrow) in the meninges. [Scale bars: 10 μm (A-C, uranyl acetate-lead citrate) and 30 μm (D-I, toluidine blue).]

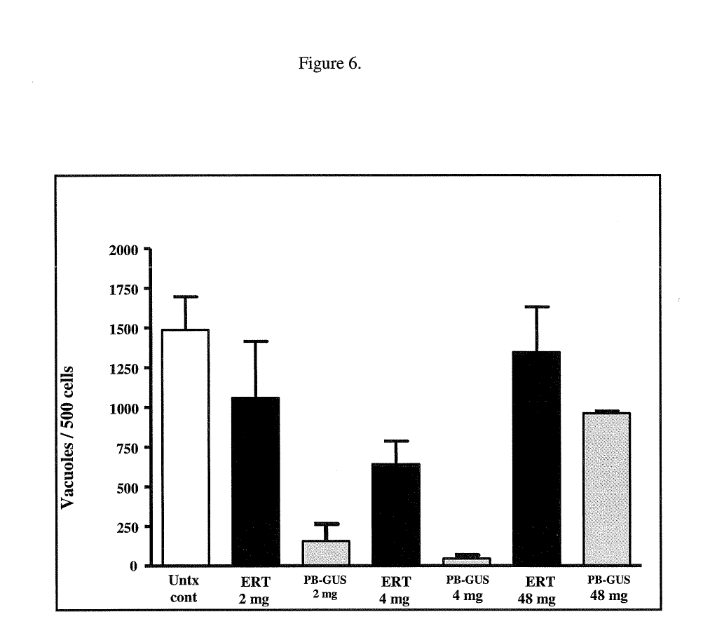

Two of the problems associated in the analysis of micrographs for the clearance of storage material in these types of experiments are: 1) that there is some inconsistency from field to field i.e. the clearance varies from one microscopic field to another; and 2) the procedure is somewhat subjective from person to person as to the amount of storage present. To address these problems, a new method was developed to quantify the storage material by counting the number of vacuoles (distended lysosomes filled with storage material) present in a total of 500 cells counted. FIG. 6 shows the results of such an analysis of the mice treated with GUS or PB-GUS.

GUS at 2 mg/kg is not very effective at reducing the number of vacuoles, though somewhat better at the higher dose of 4 mg/kg. However, PB-GUS appears to be almost completely effective at both 2 and 4 mg/kg. This analysis agrees with the conclusion drawn from the visual analysis of the images in FIG. 5.

Table 5 below summarizes the results of assessment of storage in neocortical and hippocampal neurons of untreated GUS and PB-GUS in MPS VII mice. ERT with GUS over 12 weeks with both 2 mg/kg and 4 mg/kg GUS reduced storage in neocortical neurons compared with untreated MPS VII mice (P=0.002 and P=0.003, respectively), although hippocampal neurons failed to show a morphological response to this therapy. PB-GUS at 2 mg/kg also reduced neocortical neuronal storage (P=0.001). At 4 mg/kg, the therapeutic effect of PB-GUS was even more striking (P=0.003 for 2 vs. 4 mg of PB-GUS and P<0.001 compared with untreated). In addition, there was virtually no storage in the hippocampal neurons in the three PB-GUS-treated mice available for quantitation (the CA2 region was not present in the section and was therefore not available for quantitation in two of the five PB-GUS-treated mice). These results indicate that ERT with PB-GUS is remarkably more effective than traditional GUS at clearing storage in the neocortical and especially hippocampal neurons in the MPS VII mouse. As a group, the PB-GUS-treated mice also had slightly less storage in glial and perivascular cells than the GUS-treated mice. However, the dose-dependent reduction in storage in meninges, which was moderate at 4 mg/kg, was equivalent in the PB-GUS- and the GUS-treated animals.

From the above results it is reasonable to expect that treatment of mammalian species in accordance with this invention will provide relief of lysosomal storage disease, particularly in humans particularly in the brain of humans.

TABLE 5
QUANTITATION OF LYSOSOMAL STORAGE IN NEURONS
IN CONTROL AND TREATED MPS VII MICE

Vacuoles per 500 cells

| | Neocortical | Hippocampal |
|---|---|---|
| Treatment | neurons | neurons |
| Control MPS VII | 1,956 | 692 |
| | 1,685 | 694 |
| | 1,927 | |
| GUS 2 mg/kg | 728 | 641 |
| | 744 | 674 |
| | 1,088 | |
| GUS 4 mg/kg | 1,274 | 642 |
| | 1,213 | |
| PB-GUS 2 mg | 403 | 2 |
| | 439 | |
| PB-GUS 4 mg | 73 | 0 |
| | 148 | 5 |
| | 72 | |

The following references are cited throughout this disclosure and are herein incorporated by reference. They are meant to illustrate and support the invention. Applicants reserve the right to challenge the veracity of any statements made therein.

1. Neufeld E F, Muenzer J (2001) in The Metabolic and Molecular Bases of Inherited Disease, eds Scriver C R, Beaudet A L, Sly W S, Valle D(McGraw-Hill, New York), pp 3421-3451.

2. Desnick R J (2004) Enzyme replacement and enhancement therapies for lysosomal diseases. J Inherit Metab Dis 27:385-410.

3. Brady R O, Barton N W (1996) Enzyme replacement and gene therapy for Gaucher's disease. Lipids 31:S137-S139.

4. Kakkis E D, et al. (2001) Enzyme-replacement therapy in mucopolysaccharidosis I. N Engl J Med 344:182-188.

5. Harmatz P, et al. (2006) Enzyme replacement therapy for mucopolysaccharidosis VI: A phase 3, randomized, double-blind, placebo-controlled, multinational study of recombinant human N-acetylgalactosamine 4-sulfatase (recombinant human arylsulfatase B or rhASB) and follow-on, open-label extension study. J Pediatr 148:533-539.

6. Brady R O (2006) Enzyme replacement for lysosomal diseases. Annu Rev Med 57:283-296.

7. Vogler C, et al. (2001) Murine mucopolysaccharidosis VII: Impact of therapies on the phenotype, clinical course, and pathology in a model of a lysosomal storage disease. Pediatr Dev Pathol 4:421-433.

8. Vogler C, et al. (1993) Enzyme replacement with recombinant p-glucuronidase in the newborn mucopolysaccharidosis type VII mouse. Pediatr Res 34:837-840.

9. Vogler C, et al. (1996) Enzyme replacement with recombinant p-glucuronidase in murine mucopolysaccharidosis type VII: Impact of therapy during the first six weeks of life on subsequent lysosomal storage, growth, and survival. Pediatr Res 39:1050-1054.

10. Urayama A, Grubb J H, Sly W S, Banks W A (2004) Developmentally regulated mannose 6-phosphate receptor-mediated transport of a lysosomal enzyme across the blood-brain barrier. Proc Natl Acad Sci USA 101:1 26 58-1 2663.

11. Vogler C, et al. (2005) Overcoming the blood-brain barrier with high-dose enzyme replacement therapy in murine mucopolysaccharidosis VII. Proc Natl Acad Sci USA 102:14777-14782.

12. Dunder U, et al. (2000) Enzyme replacement therapy in a mouse model of aspartylg-lycosaminuria. FASEB J 14:361-367.

13. Roces D P, et al. (2004) Efficacy of enzyme replacement therapyin a-mannosidosis mice: A preclinical animal study. Hum Mol Genet 13:1979-1988.

14. Matzner U, et al. (2005) Enzyme replacement improves nervous system pathology and function in a mouse model for metachromatic leukodystrophy. Hum Mol Genet 14:1139-1152.

15. Sly W S, et al. (2006) Enzyme therapy in mannose receptor-null mucopolysaccharidosis VII mice defines roles for the mannose 6-phosphate and mannose receptors. Proc Natl Acad Sci USA 103:1 5172-15177.

16. Ashwell G, Harford J(1982) Carbohydrate-specific receptors of the liver. Annu Rev Biochem 51:531-554.

17. Hickman S, Shapiro L J, Neufeld E F (1974) A recognition marker required for uptake of a lysosomal enzyme by cultured fibroblasts. Biochem Biophys Res Commun 57:55-61.

18. Achord D T, Brot F E, Bell C E, Sly W S (1978) Human p-glucuronidase: In vivo clearance and in vitro uptake by a glycoprotein recognition system on reticuloendothelial cells. Cell 15:269-278.

19. LeBowitz J H, et al. (2004) Glycosylation-independent targeting enhances enzyme delivery to lysosomes and decreases storage in mucopolysaccharidosis type VII mice. Proc Natl Acad Sci USA 101:3083-3088.

20. Houba P H, Boven E, Haisma H J (1996) Improved characteristics of a human p-glucuronidase-antibody conjugate after deglycosylation for use in antibody-directed enzyme prodrug therapy. Bioconjug Chem 7:606-611.

21. Wang C, Eufemi M, Turano C, Giartosio A (1996) Influence of the carbohydrate moiety on the stability of glycoproteins. Biochemistry 35:7299-7307.

22. Frankel H A, Glaser J H, Sly W S (1977) Human p-glucuronidase. I. Recognition and uptake by animal fibroblasts suggests animal models for enzyme replacement studies. Pediatr Res 11:811-816.

23. Achord D, Brot F, Gonzalez-Noriega A, Sly W, Stahl P(1977) Human p-glucuronidase. II. Fate of infused human placental p-glucuronidase in the rat. Pediatr Res 11:816-822.

24. Brot F E, Bell C E, Jr, Sly W S (1978) Purification and properties of p-glucuronidase from human placenta. Biochemistry 17:385-391.

25. Wu B M, et al. (1994) Over expression rescues the mutant phenotype of L176F mutation causing p-glucuronidase deficiency mucopolysaccharidosis in two Mennonite siblings. J Biol Chem 269:23681-23688.

26. Natowicz M R, Chi M M, Lowry O H, Sly W S (1979) Enzymatic identification of mannose 6-phosphate on the recognition marker for receptor-mediated pinocytosis of p-glucuronidase by human fibroblasts. Proc Natl Acad Sci USA 76:4322-4326.

27. Schlesinger P H, et al. (1980) The role of extrahepatictissues in the receptor-mediated plasma clearance of glycoproteins terminated by mannose or N-acetylglucosamine. Biochem J 192:597-606.

28. Banks W A (2004) Are the extracellular [correction of extracelluar] pathways a conduit for the delivery of therapeutics to the brain? Curr Pharm Des 10:1365-1370.

29. Niwa H, Yamamura K, Miyazaki J(1991) Efficient selection for high-expression transfectants with a novel eukaryotic vector. Gene 108:193-199.

30. Ulmasov B, et al. (2000) Purification and kinetic analysis of recombinant CA XII, a membrane carbonic anhydrase over expressed in certain cancers. Proc Natl Acad Sci USA 97:14212-14217.

31. Glaser J H, Sly W S (1973) p-Glucuronidase deficiency mucopolysaccharidosis: Methods for enzymatic diagnosis. J Lab Clin Med 82:969-977.

32. Lowry O H, Rosebrough N J, Farr A L, Randall R J (1951) Protein measurement with the folin phenol reagent. J Biol Chem 193:265-275.

33. Diment S, Leech M S, Stahl PD (1987) Generation of macrophage variants with 5-aza-cytidine: Selection for mannose receptor expression. J Leukocyte Biol 42:485-490.

34. Chinese Hamster Ovary Cell Line American Type Culture Collection, ATCC CRL 9618.

35. Laemmli, U.K., (1970) Nature(London) 2′27, 680-685.

What is claimed is:

1. An isolated, modified recombinant β-glucuronidase wherein the modification is having its carbohydrate moeties chemically modified so as to reduce its activity with respect to mannose and mannose 6-phosphate cellular delivery system while retaining enzymatic activity.

2. The modified β-glucuronidase of claim 1 derived from human β-glucuronidase.

3. The modified β-glucuronidase of claim 2 wherein the modification is provided by sequential treatment of human β-glucuronidase with an alkali metal periodate and an alkali metal borohydride.

4. The modified β-glucuronidase of claim 3 wherein the periodate is sodium periodate and the borohydride is sodium borohydride

5. The modified β-glucuronidase of claim 3 wherein sodium periodate is sodium-meta-periodate.

6. The modified β-glucuronidase of claim 1 in combination with a pharmaceutically acceptable excipient.

7. A method of treating a mammal afflicted with a lysosomal storage disease comprising administering to the mammal a therapeutically effective amount of an isolated, modified enzyme selected from recombinant β-glucuronidase and a lysosomal enzyme wherein the modification comprises having its carbohydrate moeties chemically modified so as to reduce its activity with respect to mannose and mannose β-phosphate cellular delivery systems while retaining enzymatic activity.

8. The method of claim 7 wherein the mammal is a human.

9. The method of claim 7 wherein the mammal is a mouse.

10. The method of claim 7 wherein the lysosomal storage disease is treated in the visceral organs of the mammal.

11. The method of claim 10 wherein at least one of the organs is the brain.

12. The method of claim 11 wherein the mammal is a human.

13. The method of claim 10 wherein the mammal is a mouse.

14. The method of claim 7 wherein the therapeutically effective amount of an isolated, modified enzyme selected from recombinant β-glucuronidase enzyme is in the range of from about 2 mg/kg to about 4 mg/kg of body weight of the mammal.

15. The method of claim 7 wherein said treatment results in clearance of about 95% of lysosomal storage from the cortical and hippocampal neurons in the brains of a mammal.

16. An isolated, modified lysosomal enzyme wherein the modification is having its carbohydrate moeties chemically modified so as to reduce its activity with respect to mannose and mannose 6-phosphate cellular delivery system while retaining enzymatic activity.

17. The modified lysosomal enzyme of claim 16 wherein the modification is provided by sequential treatment of said enzyme with an alkali metal periodate and an alkali metal borohydride.

18. The modified enzyme of claim 17 wherein the periodate is sodium periodate and the borohydride is sodium borohydride.

19. The modified enzyme of claim 18 wherein sodium periodate is sodium-meta-periodate.

20. The enzyme of claim 16 wherein the enzyme is selected from the group consisting of heparin N-sulfatase, β-hexosaminidase A, α-L-iduronidase, palmitoyl thiotransferase, α-glucosidase, N-acetyl-galactosamine-6-sulfatase, β-galactosidase and N-acetylgalactosamine 4-sulfatase.

21. The modified enzyme of claim 16 in combination with a pharmaceutically acceptable excipient.

22. A method of treating a mammal afflicted with a lysosomal storage disease comprising administering to the mammal a therapeutically effective amount of an isolated, modified lysosomal enzyme wherein the modification comprises having its carbohydrate moeties chemically modified so as to reduce its activity with respect to mannose and mannose 6-phosphate cellular delivery systems while retaining enzymatic activity.

23. The method of claim 22 wherein the mammal is a human.

24. The method of claim 22 wherein the mammal is a mouse.

25. The method of claim 22 wherein the lysosomal storage diseases is treated in the visceral organs of the mammal.

26. The method of claim 25 wherein at least one of the organs is the brain.

27. The method of claim 26 wherein the mammal is a human.

28. The method of claim 26 wherein the mammal is a mouse.

29. The method of claim 22 wherein the enzyme is selected from the group consisting of heperan N-sulfatase, β-hexosamidase A, α-L-iduronidase, palmitoyl thiotransferase, α-glucosidase, N-acetyl-galactosamine-6-sulfatase, β-galactosidase and N-acetylgalactosamine 4-sulfatase.

30. The method of claim 22 wherein the therapeutically effective amount of an isolated, modified enzyme selected from recombinant β-glucuronidase enzyme is in the range of from about 2 mg/kg to about 4 mg/kg of body weight of the mammal.

31. The method of claim 30 wherein said treatment results in clearance of about 95% of lysosomal storage from the cortical and hippocampal neurons in the brains of a mammal.


SEQUENCE LISTING (2 sequences)

SEQ ID NO: 1 - DNA, 2191 bp, Human B-glucuronidase
ggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatatgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtcgctactg

SEQ ID NO: 2 - protein, 651 aa, Human B-glucuronidase
MARGSAVAWAALGPLLWGCALGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSPFT